(19) United States
(12) Patent Application Publication
(10) Pub. No.: US 2022/0087960 A1
(43) Pub. Date: Mar. 24, 2022

(54) Oral Pharmaceutical Immediate Release Composition

(71) Applicants: Zhenhuan Zheng, San Diego, CA (US); Tien-Li Lee, San Diego, CA (US)

(72) Inventors: Zhenhuan Zheng, San Diego, CA (US); Tien-Li Lee, San Diego, CA (US)

(73) Assignee: Aardvark Therapeutics Inc., San Diego, CA (US)

(21) Appl. No.: 17/031,811

(22) Filed: Sep. 24, 2020

(51) Int. Cl. A61K 31/167; A61K 9/00; A61K 47/02; A61K 9/16; A61K 9/48

(52) U.S. Cl. CPC A61K 31/167; A61K 9/0053; A61K 47/02; A61K 9/1652; A61K 9/1676; A61K 9/1617; A61K 9/1635; A61K 9/4825; A61K 9/1623

(57) ABSTRACT

There is disclosed an oral pharmaceutical composition for the treatment of multiple diseases comprising a denatonium cation salt and a sour anion selected from the group consisting of acetate (DA), citrate (DC) tartrate (CT), maleate (DM) and combinations thereof (collectively “denatonium salt”) and pharmaceutical excipients for gastric release of the denatonium salt. There is further disclosed an oral immediate release pharmaceutical composition to substantially release an API (active pharmaceutical ingredient) in the gastric area of the GI tract formulation, wherein the API comprises an effective amount of the denatonium salt. Preferably, the oral immediate release pharmaceutical formulation comprises from about 0.5 g to about 5 g of the denatonium salt delivering a daily dose of the denatonium salt from about 20 mg to about 150 mg to a human adult.

### CROSS REFERENCE TO RELATED APPLICATIONS

This patent application is a continuation-in-part of pending U.S. patent application Ser. No. 16/004,700 filed 11 Jun. 2018, which claims priority from U.S. provisional patent application 62/665,230, filed 1 May 2018.

### TECHNICAL FIELD

The present disclosure provides an oral pharmaceutical composition for the treatment of multiple diseases comprising a denatonium cation salt and a sour anion selected from the group consisting of acetate (DA), citrate (DC) tartrate (CT), maleate (DM) and combinations thereof (collectively “denatonium salt”) and pharmaceutical excipients for gastric release of the denatonium salt. The present disclosure further provides an oral immediate release pharmaceutical composition to substantially release an API (active pharmaceutical ingredient) in the gastric area of the GI tract formulation, wherein the API comprises an effective amount of the denatonium salt. Preferably, the oral immediate release pharmaceutical formulation comprises from about 0.5 g to about 5 g of the denatonium salt delivering a daily dose of the denatonium salt from about 20 mg to about 150 mg to a human adult.

### BACKGROUND

Chemosensory signaling of nutrients plays a role in regulating appetite, digestion, and metabolism. In particular, a variety of bitter taste receptors (TAS2R) family of G-protein-coupled receptors (GPCRs) exist not only in the oral cavity, but on gut endocrine cells, human gastric smooth muscle cells, adipocytes, as well as sites in the chemoreceptor trigger zone in the medulla of the brain.

Obesity is a global pandemic that has led to serious health and socioeconomic consequences for millions of adults and children (Bluher, Nat. Rev. Endocrinol. 15 (2019) 288-298). Globally, at least 13% of adults and 7% of children are obese, but in several countries the prevalence of obesity is at least 30% of the overall population (Ng et al. Lancet 384 (2014) 766-781).

Obesity is ideally treated with dieting and physical exercise, but success rates for such programs have been observed to be low, at approximately 20%. Often, this is largely due to a strong appetite drive. which has redundant stimulatory pathways. and is difficult to overcome, as suppression of one pathway for appetite generation frequently results in upregulation of compensatory alternate pathways to invoke hunger over time. Various medications that have been commercially available confer generally modest results or have accompanying risk and side effects that are deemed intolerable by many, or both.

Anorexigenic stimulant compounds such as ephedrine, fenfluramine, and dexfenfluramine were withdrawn from the market due to associated cardiovascular safety risks. Drugs that interfere with nutrient absorption such as Orlistat, a lipase inhibitor, which blocks fat processing in the gut, results in oily stool and diarrhea. Central nervous system targeted drugs such as Sibutramine (a monoamine oxidase inhibitor), Rimonabant (a cannabinoid receptor antagonist), and others, have significant central nervous system (CNS) “off-target” effects often leading to unintended psychiatric or neurological manifestations.

Behavioral interventions for obesity, such as exercise regimens and changes in diet, often fail, and bariatric surgery is not an option for most people. Anti-obesity drugs can be effective at lowering body weight; however, they have been associated with side effects ranging from headache, nausea, and dizziness to severe psychiatric and cardiovascular events (M. O. Dietrich et al., Nat. Rev. Drug Discov. 11 (2012) 675-691). Given the enormous medical, societal, and economic burden of obesity, there is an urgent need to develop novel, safe, and effective therapeutic agents for this debilitating and potentially fatal disease.

Bitter taste receptors (TAS2Rs) comprise a family of several G-protein coupled receptors (GPCRs) that are expressed on the tongue as well as other organs, including the brain, oral cavity, lung, pancreas, and gastrointestinal mucosa (Jaggupilli et al., Mol. Cell. Biochem. 426 (2017) 137-147).

Denatonium benzoate activates to varying degrees eight human TAS2Rs (TAS2R 4, 8, 10, 13, 39, 43, 46, and 47) (Meyerhof et al., Chem. Senses 35 (2010) 157-170). In rodent obesity models, the benzoate salt of denatonium suppressed food intake and inhibited weight gain (Avau et al., PLoS One 10 (2015) e0145538; and Glendinning et al., Physiol. Behav. 93 (2008) 757-765). Further, in healthy volunteers, denatonium benzoate attenuated inter-digestive gastric motility, reduced nutrient volume tolerance, decreased hunger ratings, and increased satiation post meal after intragastric administration (Avau et al., Sci. Rep. 5 (2015) 15985; and Deloose et al., Am. J. Clin. Nutr. 105 (2017) 580-588). However, a significant side effect problem emerged with the two denatonium benzoate studies due to the aversive nature of denatonium benzoate. Therefore, there is a significant need in the art to address obesity and related disorders with an improved bitter agonist that has a safer profile. The present disclosure addresses this need.

### SUMMARY

The present disclosure is based on a finding that denatonium salts that have a sour anion have better side effect profiles seen in comparative in vivo studies versus denatonium benzoate, the only available denatonium salt and the denatonium salt reported in earlier studies.

The present disclosure provides an oral pharmaceutical composition for the treatment of multiple diseases comprising a denatonium cation salt and a sour anion selected from the group consisting of acetate (DA), citrate (DC) tartrate (CT), maleate (DM) and combinations thereof (collectively “denatonium salt”) and pharmaceutical excipients for gastric release of the denatonium salt. The present disclosure further provides an oral immediate release pharmaceutical composition to substantially release an API (active pharmaceutical ingredient) in the gastric area of the GI tract formulation, wherein the API comprises an effective amount of the denatonium salt. Preferably, the oral immediate release pharmaceutical formulation comprises from about 0.5 g to about 5 g of the denatonium salt delivering a daily dose of the denatonium salt from about 20 mg to about 150 mg to a human adult.

The present disclosure provides an oral pharmaceutical immediate gastric release pharmaceutical formulation (“oral formulation”) comprising granules which comprise a denatonium cation salt and a sour anion selected from the group consisting of acetate (DA), citrate (DC) tartrate (CT), maleate (DM) and combinations thereof (collectively “denatonium salt”) and pharmaceutical excipients for gastric release of the denatonium salt. Preferably the pharmaceutical excipients comprise talc, a cellulose and a saccharide. Preferably, the oral formulation further comprises an organic acid selected from the group consisting of acetic acid, malic acid, maleic acid, citric acid and combinations thereof. Preferably, the oral formulation further comprises from about 0.5 g to about 5 g acetic acid. More preferably, the dosage per day of the acetic acid for an adult is from about 1.5 g to about 3 g. Preferably the daily dosage of DA for an adult is from about 10 mg to about 600 mg or from about 5 mg/kg to about 50 mg/kg body weight per day. More preferably, the daily dosage of DA for an adult is from about 10 mg to about 200 mg. Most preferably, the daily dosage of DA for an adult is from about 10 mg to about 100 mg, or to achieve a concentration in the GI tract of from about 10 parts per billion to about 10 ppm. In view of the sustained release or immediate release characteristics, the daily dose of DA is once per day, twice per day or three times per day.

Further, the present disclosure provides a sustained release oral formulation comprising DA and acetic acid powder in a sustained release cellulosic and mannitol excipient formulation. Preferably the daily dosage of DA for an adult is from about 10 mg to about 600 mg. More preferably, the daily dosage of DA for an adult is from about 10 mg to about 200 mg. Most preferably, the daily dosage of DA for an adult is from about 10 mg to about 100 mg, or to achieve a concentration in the GI tract of from about 10 parts per billion to about 10 ppm. Preferably, the oral formulation comprises from about 0.01% to about 10 wt % DA and from about 10% to about 90 wt % dry acetic acid powder. Preferably, the dose administered of DA is from about 500 nmol/kg to about 4 μmol/kg. Preferably, the dose administered of DA is from about 10 mg to about 50 mg for an adult. In view of the release characteristics, the daily dose of DA is once per day, twice per day or three times per day.

The present disclosure further provides a method for effecting weight loss, comprising administering an oral pharmaceutical immediate gastric release pharmaceutical formulation (“oral formulation”) comprising granules which comprise a denatonium cation salt and a sour anion selected from the group consisting of acetate (DA), citrate (DC) tartrate (CT), maleate (DM) and combinations thereof (collectively “denatonium salt”) and pharmaceutical excipients for gastric release of the denatonium salt. Preferably the pharmaceutical excipients comprise talc, a cellulose and a saccharide. Preferably, the oral formulation further comprises an organic acid selected from the group consisting of acetic acid, malic acid, maleic acid, citric acid and combinations thereof. Preferably, the oral formulation further comprises from about 0.5 g to about 5 g acetic acid. More preferably, the dosage per day of the acetic acid for an adult is from about 1.5 g to about 3 g. Preferably, the daily dosage of DA for an adult is from about 10 mg to about 600 mg or from about 5 mg/kg to about 50 mg/kg body weight per day. More preferably, the daily dosage of DA for an adult is from about 10 mg to about 200 mg. Most preferably, the daily dosage of DA for an adult is from about 10 mg to about 100 mg, or to achieve a concentration in the GI tract of from about 10 parts per billion to about 10 ppm.

### FIGURES

FIG. 1 shows a 56 day DIO mouse weight loss study comparison (Example 3) of body weights at the indicated days. The higher dose DA group (23.1 mg/kg) showed the lowest average body weights.

FIG. 2 shows the results of body weight changes of the 56 day study in Example 3. Animals treated with 23.1 mg/kg DA showed the lowest increase in body weight over a higher dose DB group.

FIG. 3 shows the results of serum insulin at the end of the 56 day study in Example 3. Serum insulin in the 23.1 mg/kg DA group was close to baseline value (i.e., pre-treatment) and noticeably lower compared to the vehicle-treated group.

FIG. 4 shows that there was no statistical difference in serum HBA1c levels among all the experimental groups in Example 3.

FIG. 5 shows cumulative food consumption over 24 hours for the single day rat study described in Example 4.

FIG. 6 shows mean absolute body weight change during 56-day treatment period in DIO mice from Example 6.

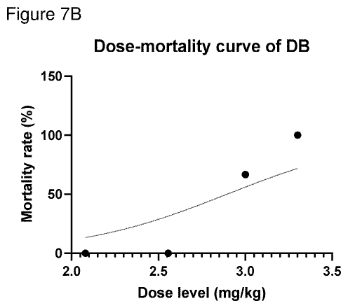

FIGS. 7A and 7B show dose-mortality curves of DA and DB from Example 7.

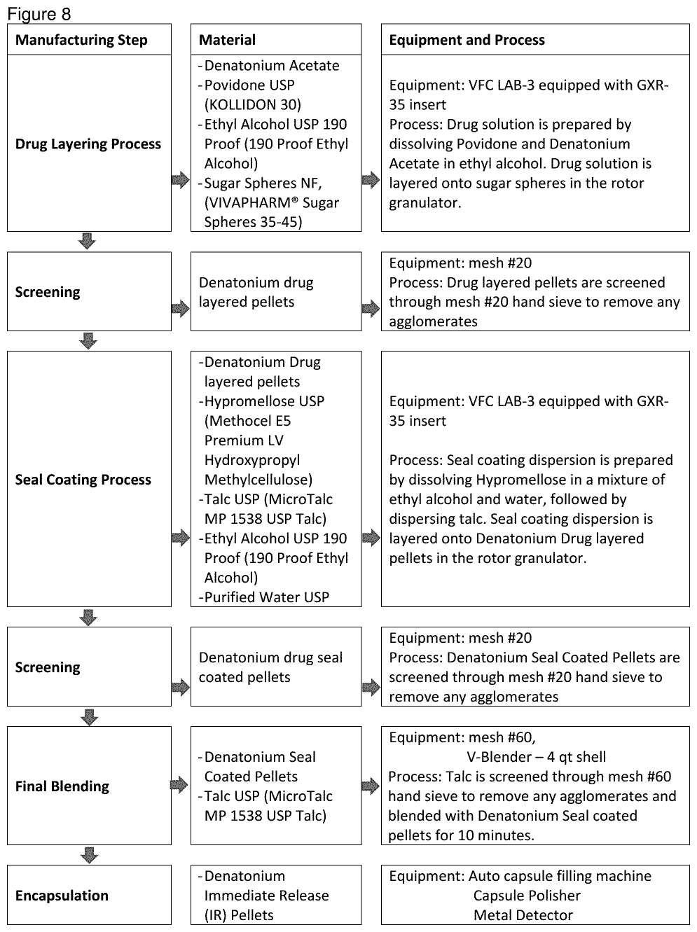

FIG. 8 shows a drug product/formulation flow diagram.

### DETAILED DESCRIPTION

The present disclosure is based on a surprising finding that the anti-obesity effects of denatonium salts are superior (both safety and efficacy) with a sour-tasting anion (acetate, citrate, tartrate and maleate) using in vitro and in vivo models of obesity. The objectives of our study were to determine the effects of denatonium salts with a sour-tasting anion on food and water consumption, body weight control.

In a short-term food intake inhibition study, in Sprague Dawley rats the doses of DA administered are 7.5, 15, 30, and 60 mol/kg. The corresponding human equivalent doses (HED) are 1.2, 2.4, 4.9, 9.7 mol/kg, respectively. In a longer-term food intake inhibition study in C57BL/6NTac mice the dose of DA is 60 mol/kg. The corresponding HED is 4.9 mol/kg. As a background, according to Avau et al. (Sci. Rep. (2015) 5:15985), oral administration of only denatonium benzoate (DB), a related salt to DA, at 60 μmol/kg (26.8 mg/kg) significantly inhibited gastric emptying rate in normal C57BL/6 mice. In another study, treatment with 60 μmol/kg DB (26.8 mg/kg) once daily induced a decrease in body weight of C57BL/6 DIO mice during a 28-day period, as compared to vehicle. According to Avau et al., healthy volunteers receiving 1 μmol/kg DB showed decreased nutrient volume tolerance and increased satiation. Therefore, the disclosed formulation provides a dose of DA from about 500 nmol/kg to about 10 mol/kg, which corresponds to from about 10 mg to about 230 mg for a human adult.

TABLE 1
Denatonium salts

| name | chemical structure |
|---|---|
| Denatonium benzoate (DB) | 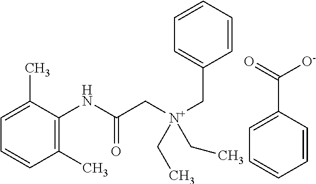 [SMILES: CC[N+](CC)(CC(=O)Nc1c(C)cccc1C)Cc1ccccc1 ; O=C([O-])c1ccccc1] |
| Denatonium acetate | 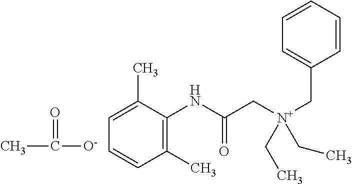 |

Denatonium Benzoate (DB)
IUPAC Name: benzyl-[2-(2,6-dimethylanilino)-2-oxoethyl]-diethylazanium benzoate
Molecular Formula: C₂₈H₃₄N₂O₃
Molecular Mass: 446.581 g/mol
CAS Number: 3734-33-6
ChemSpider ID:
Denatonium, usually available as denatonium benzoate (under trade names such as BITTERANT-b, BITTER+PLUS, Bitrex or Aversion). It is used as an aversive agent (bitterants) to prevent inappropriate ingestion. Denatonium benzoate is used in denatured alcohol, antifreeze, nail biting preventions, respirator mask fit-testing, animal repellents, liquid soaps, and shampoos. It is not known to pose any long-term health risks.

A treatment that could utilize a compound with low inherent toxicity to trigger extra-oral bitter receptors in the gut, brain, and other regions such as adipocytes provides a relatively safe means to decrease appetite and increase satiety selectively without the “off-target” CNS effects or GI disturbance typical of other obesity medications.

A clinical use for a combination orally ingested tablet or pill containing DA in combination with an organic acid, such as acetic acid, beyond obesity is Prader-Willi Syndrome. Among the key hallmarks of this genetic disorder is a constant hunger drive and a lack of sense of satiety even after eating copious amounts of food. Therefore, the present disclosure provides a method for treating Prader-Willi Syndrome (PWS) comprising an anti-obesity oral formulation comprising (a) denatonium acetate (DA), (b) an organic acid selected from the group consisting of acetic acid, malic acid, maleic acid, citric acid and combinations thereof; and (c) pharmaceutical excipients to facilitate a sustained release during transit through the GI tract.

### Example 1

This example describes a method for formulating a Denatonium Acetate/Acetic Acid Release Tablet, 44.6 mg/500 mg.

TABLE 2

| | Per dose¹, | Quantity, |
|---|---|---|
| Ingredient | mg | kg |
| Denatonium Acetate | 44.6 | 5.575 |
| Acetic Acid, NF (36.5% w/w) | 1370 | 171.25 |
| Microcrystalline Cellulose | 100 | 12.5 |
| Mannitol | 80 | 10 |
| Polyvinyl Pyrrolidone 30 (PVP 30) | 38 | 4.75 |
| Magnesium Stearate | 4 | 0.5 |
| Ethylcellulose aqueous dispersion | 786.7 | 98.34 |
| (Aquacoat ECD 30, FMC). | | |
| Dibutyl Sebacate | 59 | 7.375 |

¹A dose can be from one to five tablets.

Add microcrystalline cellulose (Avicel PH101), denatonium acetate, PVP 30 (half quantity) and mannitol to a 10 cubic feet V-blender and mix for 10 minutes. Transfer the blend to a high shear granulator and start granulating with a controlled spray rate of acetic acid (half quantity) at 800 g/minute. After granulation, the wet granules are removed and placed in a tray dryer controlled at 50° C. for a period until the final moisture content is below 2% w/w. The dried granules are subsequently passed through a Fitzmill equipped with 18 mesh screen. The milled granules are then placed back to the same high shear granulator and add the remaining half of the PVP 30 and again granulating with the remaining half of the acetic acid. The wet granules are removed and dried at 50° C. until the moisture content is below 2%. The dried granules are milled in a Fitzmill with 18 mesh screen, and then mixed with Magnesium Stearate in a 10 cubic feet V-blender for 5 minutes and the final blends are compressed in a tablet press with target 786.6 mg weight and 10 kp hardness (Uncoated Tablets).

The coating solution is prepared by dispersing dibutyl sebacate in the Aquacoat ECD 30 dispersion and gently mix for 1 hour. The Uncoated Tablets are loaded in a pan coater and sprayed with the Coating Solution at a controlled spray rate of 80 g/min. Continue drying for 30 minutes after the coating is complete.

### Example 2

This example describes a method of Denatonium Acetate/Acetic Acid Immediate Release Tablet, 22.3 mg/250 mg.

TABLE 3

| Ingredient | Per dose², mg | Quantity, kg |
|---|---|---|
| Denatonium Acetate | 22.3 | 4.46 |
| Acetic Acid, NF (36.5% w/w) | 685 | 137 |
| Microcrystalline Cellulose | 100 | 20 |
| Mannitol | 90.2 | 18.04 |
| Polyvinyl Pyrollidone 30 (PVP 30) | 25 | 5 |
| Magnesium Stearate | 2.5 | 0.5 |

²A dose can be from one to five tablets.

Add microcrystalline cellulose (Avicel PH101), denatonium acetate, PVP 30 (half quantity) and mannitol to a 10 cubic feet V-blender and mix for 10 minutes. Transfer the blend to a high shear granulator and start granulating with a controlled spray rate of acetic acid (half quantity) at 800 g/minute. After granulation, the wet granules are removed and placed in a tray dryer controlled at 50° C. for a period until the final moisture content is below 2% w/w. The dried granules are subsequently passed through a Fitzmill equipped with an 18 mesh screen. The milled granules are then placed back to the same high shear granulator and add the remaining half of the PVP 30 and again granulating with the remaining half of the acetic acid. The wet granules are removed and dried at 50° C. until the moisture content is below 2%. The dried granules are milled in a Fitzmill with 18 mesh screen and then mixed with Magnesium Stearate in a 10 cubic feet V-blender for 5 minutes and the final blends are compressed in a tablet press with target 500 mg weight and 10 kp hardness.

### Example 3

This example shows an acute and a chronic in vivo study comparing the weight loss properties of DA versus DB (denatonium benzoate), two salts having the same cation and different anions. The 56-day study determined the behavioral effects of bitter taste receptor agonists denatonium acetate (DA) compared to denatonium benzoate (DB) in a diet-induced obesity (DIO) mouse model. The animals were acclimated in a vivarium for at least 3 days, maintained on a standard chow diet, 12:12 light/dark cycle and group housed 2-3 in heap-filtered cages. The study duration was a 3-5 day acclimation period+28 day study period and 2-3 day testing period after study. There were two DA dosages of 2.9 and 23.1 mg/kg BID (3.1 and 60 mol/kg BID), 26.8 mg/kg DB BID and distilled water control vehicle with DA and DB made up in distilled water. The mice were C57BL/6NTad mice at least 12 weeks in age and 15 mice per group (low dose DA, higher dose DA, high dose DB and control). There were gross observations each day, and body weight measurements for each animal on Days 0, 1, 4, 7, 9, 11, 14, 16, 18, 21, 23, 25, 28, 30, 32, 34, 36, 39, 41, 43, 46, 48, 50, 53, and 56. Food intake was measured on Days 0, 7, 14, 21, 28, 35, 42, 49, and 56. Metabolic biomarkers (blood glucose, blood insulin, blood HbA1c) were measured at the beginning and end of the study. The DA, DB or distilled water control was administered per ostium gavage (PO) at a volume of 5 mL/kg body weight.

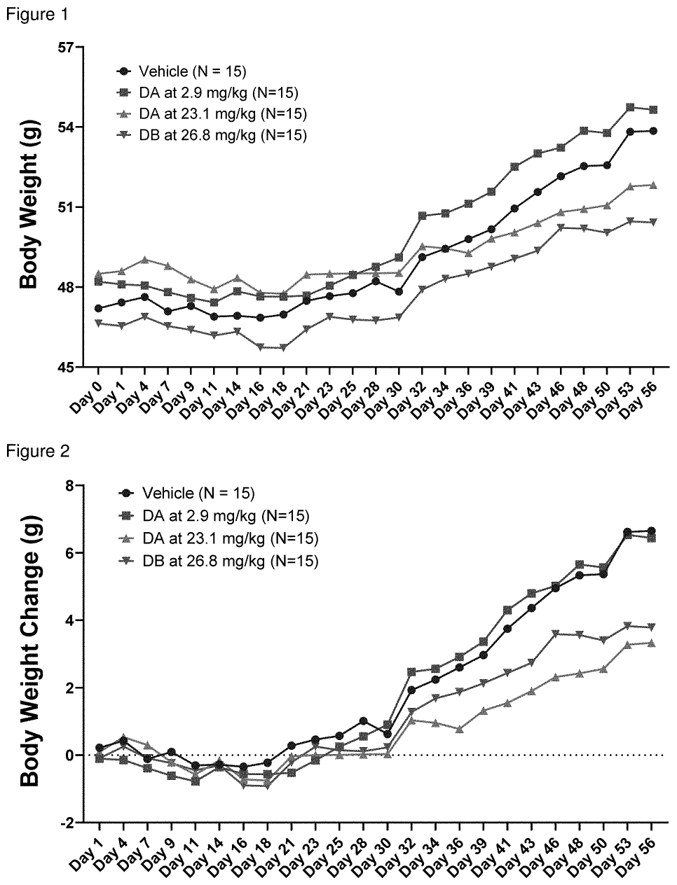

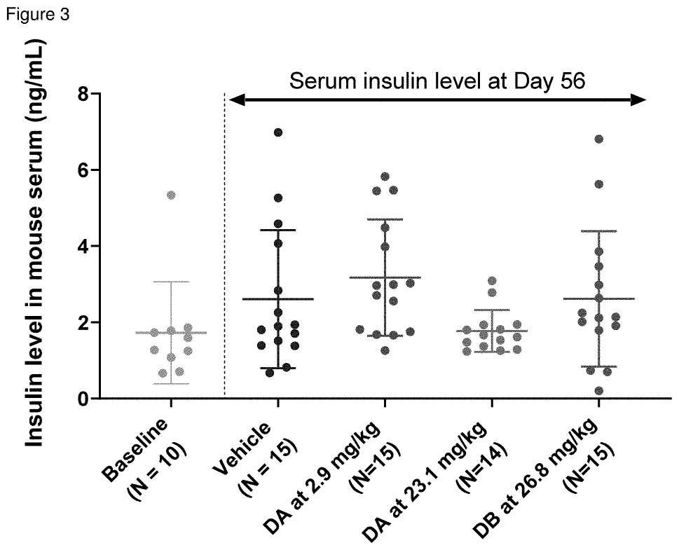

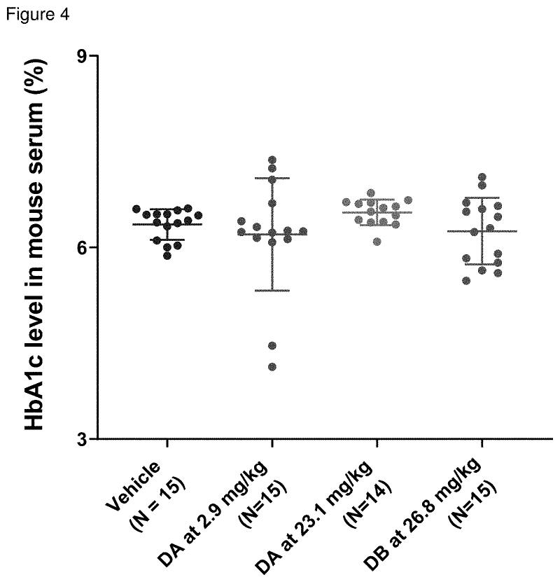

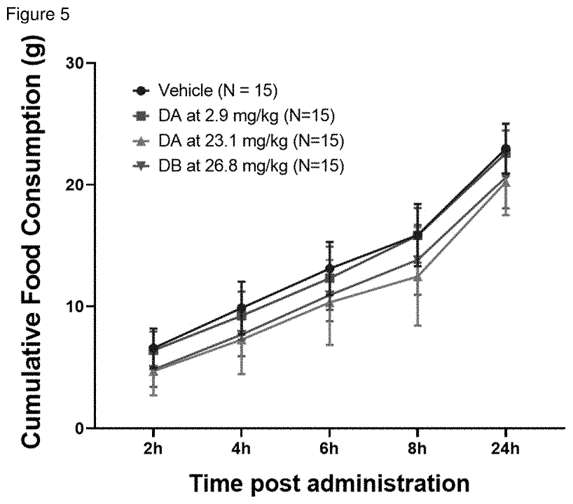

The results are shown in FIGS. 1-4 showing weight loss improvement of higher dose DA over higher dose DB.

### Example 4

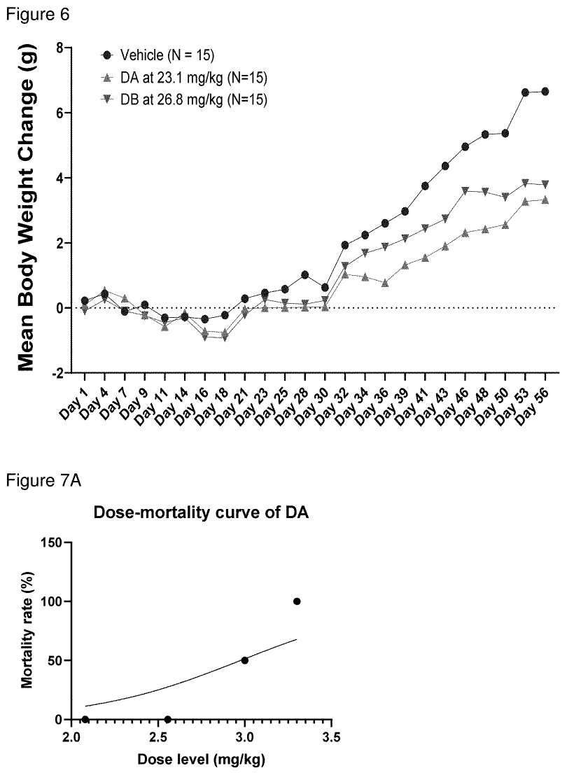

This example provides a 24 hour study comparing DA to DB is rats (male Sprague Dawley, Charles River) over a 24 hour period. The 5 groups of 15 rats each were vehicle controlled distilled water gavage QID, DB at a dose of 26.8 mg/kg gavage QID, DA low dose 2.9 mg/kg gavage QD, and DA high dose 23.1 mg/kg gavage QD. Food intake at 2 hr, 4 hr, 6 hr, 8 hr and 24 hr after administration was measured. The results of cumulative food consumption over 24 hour time are shown in FIG. 5 are that there was a significant main effect of drug treatment on cumulative food consumption with higher dose DA group having the largest impact.

### Example 5

This example describes the synthesis of denatonium acetate (DA).

Step 1: Synthesis of Denatonium Hydroxide from Lidocaine

To a reflux apparatus add 25 g of lidocaine, 60 ml of water and 17.5 g of benzyl chloride with stirring and heating in 70-90° C. The solution needs to be heated and stirred in the before given value for 24 h, the solution needs to be cooled down to 30° C. The unreacted reagents are removed with 3×10 mL of toluene. With stirring dissolve 65 g of sodium hydroxide into 65 mL of cold water and add it to the aqueous solution with stirring over the course of 3 h. Filter the mixture, wash with some water and dry in open air. Recrystallize in hot chloroform or hot ethanol.

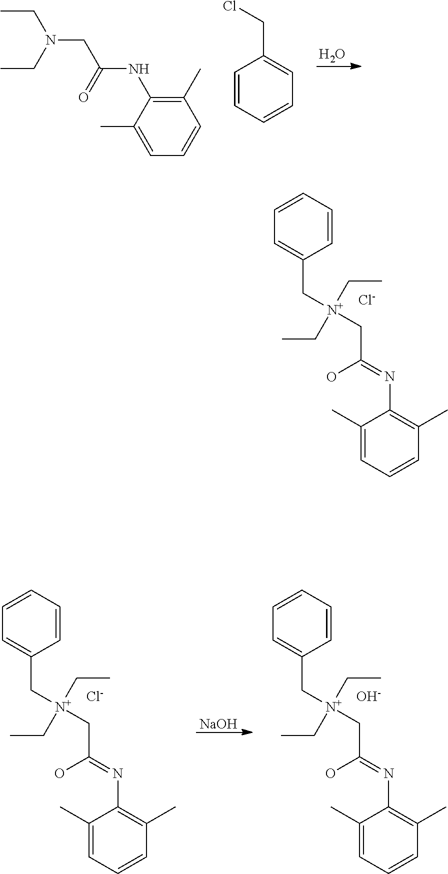

Step 2: Preparation of Denatonium Acetate from Denatonium Hydroxide.

To a reflux apparatus 10 g of denatonium hydroxide (MW: 342.475 g/mol, 0.029 mol), 20 mL of acetone, and 2 g of acetic acid glacial (0.033 mol) dissolved in 15 mL of acetone is added, the mixture is stirred and heated to 35° C. for 3 h. Then evaporated to dryness and recrystallized in hot acetone.

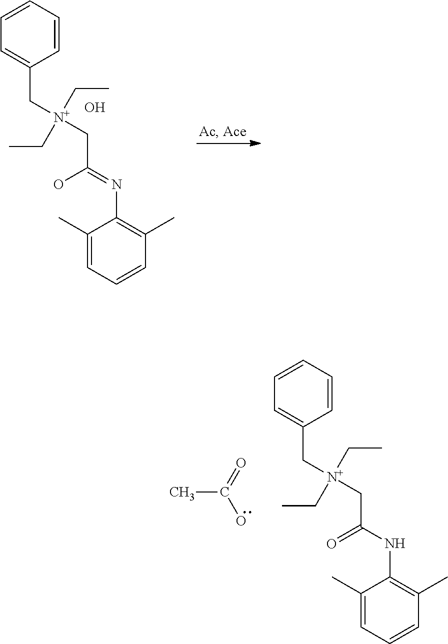

### Example 6

This example compares efficacy of DA versus DB in food inhibition and body weight control. As a background, according to Avau et al., Sci. Rep. (2015) 5:15985, oral administration of 26.8 mg/kg of DB significantly inhibited gastric emptying rate in normal C57BL/6 mice. In our first in vivo study, 45 male SD rats (purchased from Envigo at 8-10 weeks of age) were divided into three groups (15 in each group), which were administered a single oral dose of vehicle (distilled water), 26.8 mg/kg of DB, or 23.1 mg/kg of DA respectively, with a 24-hour observational period, to compare the efficacy of DB versus DA in reducing food intake. Table 4. Mean cumulative food intake during 24-h observational period

| | Mean cumulative food intake (gram) | | | | |
|---|---|---|---|---|---|
| Group | 0-2 h | 0-4 h | 0-6 h | 0-8 h | 0-24 h |
| DA 23.1 mg/kg | 4.7 | 7.3 | 10.3 | 12.5 | 20.2 |
| (N = 15) | | | | | |
| DB 26.8 mg/kg | 4.8 | 7.7 | 10.9 | 13.8 | 20.6 |
| (N = 15) | | | | | |
| Vehicle | 6.6 | 9.9 | 13.1 | 15.9 | 23.0 |
| (N = 15) | | | | | |

The mean cumulative food intake during 24-h observational period is presented in Table 4. Administration with DB or DA reduced cumulative food intake during all the indicated time intervals as compared with vehicle. Additionally, a greater food intake reduction was observed with dosing with 23.1 mg/kg of DA than with 26.8 mg/kg of DB, although the molar dose of DA was even lower than DB (57.4 μmol/kg vs. 60 mol/kg). Therefore, these data show that DA has a stronger efficacy than DB in food intake reduction on the basis of a different anion for the salt.

In another published article, treatment with 26.8 mg/kg of DB once daily induced a decrease in body weight of C57BL/6 diet-induced obese (DIO) mice during a 28-day period, as compared to vehicle (Avau et al. 2, PLoS One. 2015; 10(12):e0145538). In a second in vivo study, 45 male C57BL/6N DIO mice (purchased from Envigo at 18 weeks of age, fed with high-fat diet) were divided into three groups (15 in each group), which were orally administered vehicle (distilled water), 26.8 mg/kg of DB, or 23.1 mg/kg of DA respectively, twice daily (BID), with a 56-day treatment period to compare the efficacy of DB versus DA in food intake reduction and body weight control. Briefly, once weekly, the food weight for each cage was recorded at 0 hour and then 24 hours later, permitting calculation of food consumption for that 24-hour interval. In addition, starting from Day 0, the mice were weighed three times weekly (every 2-3 days).

The mean food consumption per animal for 24-hour intervals at indicated measurement days during the treatment period are shown in Table. Notably, mice dosed with 23.1 mg/kg DA exhibited nominally decreased food consumption compared to vehicle-dosed mice; this effect was seen throughout the study. Lower food consumption was also seen on Days 0, 7, 28, 35, 42 and 49 in animals dosed with 26.8 mg/kg DB (compared to mice dosed with vehicle), but this was not the case on Days 14, 21, and 56. And on all indicated measurement days except for Day 42, food consumption was less in animals treated with 23.1 mg/kg of DA than in those treated with 26.8 mg/kg of DB.

TABLE 5
Mean food consumption per animal for 24-h interval at indicated
measurement days

| | Mean food consumption per animal for 24-h interval (gram) | | | | | | | | |
|---|---|---|---|---|---|---|---|---|---|
| | Day | Day | Day | Day | Day | Day | Day | Day | Day |
| Group | 0 | 7 | 14 | 21 | 28 | 35 | 42 | 49 | 56 |
| DA | 2.2 | 1.7 | 1.9 | 2.3 | 2.0 | 2.4 | 2.0 | 2.3 | 1.7 |
| 23.1 | | | | | | | | | |
| mg/kg | | | | | | | | | |
| DB | 2.5 | 1.8 | 2.6 | 2.9 | 2.5 | 2.5 | 2.0 | 2.4 | 2.5 |
| 26.8 | | | | | | | | | |
| mg/kg | | | | | | | | | |
| Vehicle | 2.6 | 2.2 | 2.6 | 2.9 | 2.7 | 2.9 | 2.7 | 3.2 | 2.5 |

The mean absolute body weight change (in grams) and normalized body weight change (% of baseline) during 56-day treatment period of the three treatment groups are presented in Error! Reference source not found. and Table 6.

TABLE 6
Mean normalized body weight change (% of baseline) during 56-day treatment period in DIO mice

| Group | Mean normalized body weight (% of baseline) on indicated study days | | | | | | | | | | | |
|---|---|---|---|---|---|---|---|---|---|---|---|---|
| | 0 | 106.7 | 4 | 7 | 9 | 11 | 14 | 16 | 18 | 21 | 23 | 25 |
| DA | 100.0 | 108.4 | 101.1 | 100.6 | 99.5 | 98.8 | 99.6 | 98.5 | 98.4 | 99.9 | 99.9 | 100.0 |
| 23.1 mg/kg | | | | | | | | | | | | |
| DB | 100.0 | 114.2 | 100.5 | 99.7 | 99.4 | 99.0 | 99.3 | 98.0 | 97.9 | 99.5 | 100.5 | 100.3 |
| 26.8 mg/kg | | | | | | | | | | | | |
| Vehicle | 100.0 | 100.4 | 100.9 | 99.7 | 100.1 | 99.3 | 99.3 | 99.1 | 99.4 | 100.5 | 100.8 | 101.1 |
| | 28 | 30 | 32 | 34 | 36 | 39 | 41 | 46 | 49 | 51 | 53 | 56 |
| DA | 100.0 | 100.0 | 102.0 | 101.9 | 101.5 | 102.6 | 103.1 | 103.9 | 104.7 | 104.9 | 105.2 | 106.7 |
| 23.1 mg/kg | | | | | | | | | | | | |
| DB | 100.2 | 100.5 | 102.8 | 103.6 | 104.1 | 104.7 | 105.3 | 106.0 | 107.8 | 107.8 | 107.4 | 108.4 |
| 26.8 mg/kg | | | | | | | | | | | | |
| Vehicle | 102.0 | 101.2 | 104.0 | 104.7 | 105.5 | 106.2 | 107.9 | 109.2 | 110.5 | 111.3 | 111.4 | 114.2 |

Feeding with high-fat diet induced body weight gain in all three experimental groups. However, treatment with 26.8 mg/kg of DB or 23.1 mg/kg of DA led to less body weight gain as compared to vehicle treatment. Particularly, through Day 34 to Day 56, body weight gain was less in animals treated with 23.1 mg/kg of DA than in those treated with 26.8 mg/kg of DB. Based on these data, DA has a stronger efficacy than DB not only in food intake reduction but also in body weight control on the basis of a different anion for the salt.

### Example 7

This example shows the maximum tolerated dose of two denatonium salts, denatonium benzoate (DB, molecular weight: 446.58 g/mol) that is commercially available and denatonium acetate (monohydrate) (DA, molecular weight (MW): 402.53 g/mol) that Aardvark Therapeutics had synthesized under GMP conditions pursuant to a supply contract. The drugs were administered to Sprague Dawley rats with a 14-day observational period. Twenty-four Male Sprague Dawley (SD) rats and 24 female SD rats were purchased from Envigo at 8-10 weeks of age. The DA group had four dose levels (120, 360, 1000, and 2000 mg/kg, single administration by oral gavage), 3 rats per sex, 6 animals in total per dose level; and the DB group: four dose levels (120, 360, 1000, and 2000 mg/kg, single administration by oral gavage), 3 rats per sex, 6 animals in total per dose level. The estimated median lethal dose (LD₅₀) was determined by a nonlinear regression [model: Y=100/(1+10\hat{ }(LogEC50-X)), Hill slope=1.0) to calculate LD₅₀. The mortality rates at all dose levels in the two experimental groups are presented in Table

TABLE 7
Mortality rates at all dose levels of DA and DB

| | | | | No. of animals | |
|---|---|---|---|---|---|
| | Dose level | | No. of | died within | Mortality |
| Drug | (mg/kg) | Sex | animals | 24 h post dosing | rate |
| Denatonium | 120 | Male | 3 | 0 | 0% |
| Acetate | | Female | 3 | 0 | |
| | 360 | Male | 3 | 0 | 0% |
| | | Female | 3 | 0 | |
| | 1000 | Male | 3 | 0 | 50% |
| | | Female | 3 | 3 | |
| | 2000 | Male | 3 | 3 | 100% |
| | | Female | 3 | 3 | |
| Denatonium | 120 | Male | 3 | 0 | 0% |
| Benzoate | | Female | 3 | 0 | |
| | 360 | Male | 3 | 0 | 0% |
| | | Female | 3 | 0 | |
| | 1000 | Male | 3 | 1 | 66.7% |
| | | Female | 3 | 3 | |
| | 2000 | Male | 3 | 3 | 100% |
| | | Female | 3 | 3 | |

Although the MTD (maximum tolerated dose) was the same (360 mg/kg) for both DA and DB in rats, dosing with 1000 mg/kg DA resulted in lower mortality rate as compared with the same dose of DB (50% vs. 66.7%). Therefore, these data show that DA is a safer drug than DB on the basis of a different anion for the salt.

The dose-mortality curves of DA and DB are shown in Error! Reference source not found. A and 7B. The estimated LD₅₀ values of DA and DB and fitting parameters are presented in Error! Reference source not found. The estimated LD₅₀ of DA is higher than that of DB (945 mg/kg vs. 784 mg/kg) with the similar goodness-of-fit parameters.

TABLE 8
The estimated LD50 and fitting parameters

| | | | Goodness of Fit | |
|---|---|---|---|---|
| | | 95% CI | Degrees | |
| | Estimated | (profile likelihood) | of | |
| Drug | LD₅₀ | of LD₅₀ | Freedom | R-squared |
| Denatonium | 945 mg/kg | 149.3 to 9263 mg/kg | 3 | 0.721 |
| Acetate | | | | |
| Denatonium | 784 mg/kg | 111.8 to 7105 mg/kg | 3 | 0.724 |
| Benzoate | | | | |

Therefore, DA is a safer drug than DB on the basis of a different anion for the salt.

### Example 8

This example provides an immediate release 50 mg granule formulation of denatonium acetate monohydrate (DA) as a free base as an immediate gastric release oral pharmaceutical formulation.

TABLE 9
shows qualitative and quantitative formulation composition of DA.

| | | | | | Limits based on IID | |
|---|---|---|---|---|---|---|
| | | | | DA capsule- | Max Potency | |
| | Quality | | Quantity | 50 mg | for Unit | |
| Ingredient | Standard | Function | (%) w/w | (mg/cap) | Dose (mg) | Reference |
| Denatonium | In-house | API | 23.55 | 59.03 | N/A | N/A |
| acetate | | | | (20 mg | | |
| monohydrate | | | | Denatonium | | |
| | | | | base) | | |
| Povidone | USP | Binder | 2.36 | 5.90 | 61.5 | Oral - |
| (KOLLIDON | | | | | | Capsule |
| 30) | | | | | | |
| Sugar Spheres | NF | Substrate | 68.85 | 172.57 | 314.13 | Oral - |
| (VIVAPHARM ® | | | | | | Capsule |
| Sugar Spheres | | | | | | |
| 35-45) | | | | | | |
| Hypromellose | USP | Binder | 3.64 | 9.14 | 150 | Oral - |
| (Methocel E5 | | | | | | Capsule |
| Premium LV | | | | | | |
| Hydroxypropyl | | | | | | |
| Methylcellulose) | | | | | | |
| Talc | USP | Anti- | 1.09 | 2.74 | 14 | Oral - |
| (MicroTalc | | tacking | | | | Capsule, |
| MP 1538 | | agent | | | | coated |
| USP Talc) | | | | | | pellets |
| Talc | USP | Flow | 0.50 | 1.25 | 284.38 | Oral - |
| (extra granular) | | aid | | | | Capsule |
| (MicroTalc | | | | | | |
| MP 1538 | | | | | | |
| USP Talc) | | | | | | |
| Total weight of beads | | | | 250.62 | N/A | N/A |
| Hard Gelatin | USP | Capsule | N/A | 73.3 | 107 | Oral - |
| Capsule | | shell | | | | Capsule |
| Shells; Cap: | | | | | | |
| White | | | | | | |
| Opaque: | | | | | | |
| Body: White | | | | | | |
| Opaque; | | | | | | |
| Size: 1 | | | | | | |

| | | | |
|---|---|---|---|
| Total weight of Filled Capsule | 323.9 | N/A | N/A |

IID, the Inactive Ingredient Database; API, active pharmaceutical ingredient; USP, the US Pharmacopeia; NF, the National Formulary

*Solvents such as Ethyl Alcohol USP 190 Proof (190 Proof Pure Ethyl Alcohol) and purified water (USP) were used for the preparation of drug solution and seal coating dispersion, but are removed during the manufacturing process.

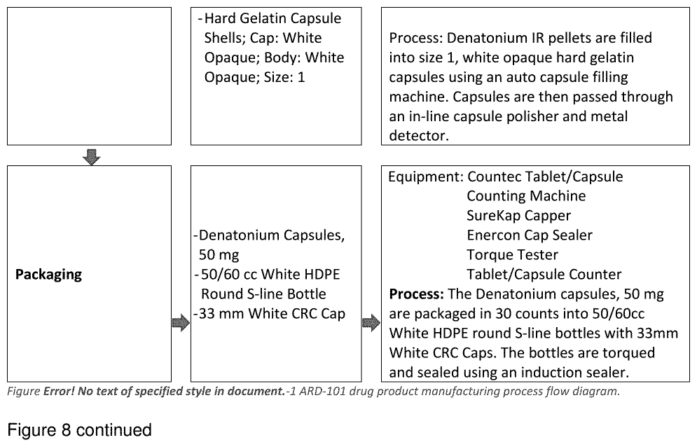

A schematic of the formulation process is shown in FIG. 8.

The detailed manufacturing steps are described below.

1. Drug Layering Process—Drug Layered Pellets

Drug layering process was performed in a Fluid bed granulator equipped with the rotor insert (rotor granulator). Drug solution was prepared by solubilizing Povidone K30 (Kollidon 30) and Denatonium Acetate in ethyl alcohol. The drug solution was sprayed tangentially on to the bed of sugar spheres (35/45 mesh) moving in a circular motion in the rotor granulator. The final drug loaded pellets were then dried for ten (10) minutes in the rotor granulator, discharged and screened through a #20 mesh. 2. Seal Coating Process—Seal coated pellets

Seal coating dispersion was prepared by separately dissolving Hypromellose ES in a mixture (1:1) of ethyl alcohol and purified water until a clear solution was obtained. The remaining quantity of ethyl alcohol was then added to the above solution followed by talc. The dispersion was mixed for 20 minutes to allow for uniform dispersion of talc. The seal coating dispersion was sprayed tangentially on to the drug loaded pellets to achieve 5% weight gain. The seal coated pellets were then dried for five (5) minutes in the rotor granulator, discharged and dried further in a tray dryer/oven at 55° C. for 2 hours. The seal coated pellets were then screened through a #20 mesh.

3. Final Blending—Denatonium Immediate Release (IR) Pellets

The seal coated pellets were blended with talc screened through mesh #60 using a V-Blender for ten (10) minutes and discharged. The blended seal coated beads, Denatonium IR Pellets, were used for encapsulation.

4. Encapsulation—Denatonium Capsules, 50 mg

The Denatonium IR pellets, 50 mg, were filled into size 1, white opaque hard gelatin capsules using an auto capsule filling machine. Capsules were then passed through an in-line capsule polisher and metal detector. In-process controls for capsule weight and appearance was performed during the encapsulation process. Acceptable quality limit (AQL) sampling and testing was performed by Quality Assurance (QA) on a composite sample during the encapsulation process. Finished product composite sample was collected and analyzed as per specification for release testing.

5. Packaging—Capsules, 50 mg-30 Counts

The 50 mg capsules were packaged in 30 counts into 50/60 cc White HDPE round S-line bottles with 33 mm White CRC Caps. The bottles were torqued and sealed using an induction sealer.

We claim:

1. An oral pharmaceutical composition comprising a denatonium cation salt and a sour anion selected from the group consisting of acetate (DA), citrate (DC) tartrate (CT), maleate (DM) and combinations thereof (collectively “denatonium salt”) and pharmaceutical excipients for gastric release of the denatonium salt.

2. An oral immediate release pharmaceutical composition to substantially release an API (active pharmaceutical ingredient) in the gastric area of the GI tract formulation, wherein the API comprises an effective amount of the denatonium salt.

3. The oral immediate release pharmaceutical formulation of claim 1 comprising from about 0.5 g to about 5 g of the denatonium salt delivering a daily dose of the denatonium salt from about 20 mg to about 150 mg to a human adult.

4. An oral pharmaceutical immediate gastric release pharmaceutical formulation (“oral formulation”) comprising granules which comprise a denatonium cation salt and a sour anion selected from the group consisting of acetate (DA), citrate (DC) tartrate (CT), maleate (DM) and combinations thereof (collectively “denatonium salt”) and pharmaceutical excipients for gastric release of the denatonium salt, wherein the pharmaceutical excipients comprise talc, a cellulose and a saccharide.

6. The oral formulation of claim 4, wherein the oral formulation further comprises an organic acid selected from the group consisting of acetic acid, malic acid, maleic acid, citric acid and combinations thereof.

7. The oral formulation of claim 4, wherein the oral formulation further comprises from about 0.5 g to about 5 g acetic acid.

8. The oral formulation of claim 4, wherein the daily dosage of DA for an adult is from about 10 mg to about 600 mg or from about 5 mg/kg to about 50 mg/kg body weight per day.

9. The oral formulation of claim 4, wherein the daily dosage of DA for an adult is from about 10 mg to about 200 mg.

10. The oral formulation of claim 4, wherein the daily dosage of DA for an adult is from about 10 mg to about 100 mg, or to achieve a concentration in the GI tract of from about 10 parts per billion to about 10 ppm.
